(19) United States
(12) Patent Application Publication
(10) Pub. No.: US 2020/0353010 A1
(43) Pub. Date: Nov. 12, 2020

(54) Therapy For Myotonic Dystrophy Type 1 Via Genome Editing of the DMPK Gene

(71) Applicant: Guangbin Xia, Albuquerque, NM (US)

(72) Inventor: Guangbin Xia, Albuquerque, NM (US)

(73) Assignee: STC.UNM, Albuquerque, NM (US)

(21) Appl. No.: 15/733,107

(22) PCT Filed: Nov. 16, 2018
(86) PCT No.: PCT/US18/61511

§ 371 Date: May 18, 2020

Related U.S. Application Data
(60) Provisional application No. 62/587,514, filed on Nov. 17, 2017.

(51) Int. Cl. A61K 35/34; A61K 35/15; C12N 5/077; A61K 9/00; C12N 15/86; C12N 7/00

(52) U.S. Cl. CPC A61K 35/34; A61K 35/15; C12N 5/0658; A61K 48/00; C12N 15/86; C12N 7/00; A61K 9/0019

(57) ABSTRACT

Methods and mechanisms for treating and/or alleviating myotonic dystrophy type 1 (DM1 by editing the DMPK gene. Editing of the DMPK gene may take place in vivo, or may involve ex vivo correction followed by implantation of genome-corrected cells.

### CROSS-REFERENCE TO RELATED APPLICATIONS

The following application claims benefit of U.S. Provisional Application No. 62/587,514, filed Nov. 17, 2017, which is hereby incorporated by reference in its entirety.

### STATEMENT REGARDING GOVERNMENT SPONSORED RESEARCH

This invention was made with Government support under Grant No. K08 AR064836 awarded by NIH/NIAMS The U.S. Government has certain rights in this invention.

### BACKGROUND

Myotonic dystrophy (Dystrophia Myotonica, DM) type 1 (DM1) is an autosomal dominant monogenic neurodegenerative disorder. It is the most common muscular dystrophy in adult with a prevalence of DM1 is 8-10/100,000^{1,2} and its congenital form, congenital myotonic dystrophy(CDM), has an incidence of 2.1 per 100,000 live births³. DM1 is a multisystemic and fetal disease. Adult classic DM1 occurs as a progressive and debilitating clinical course. Patients suffer from progressive muscle wasting, myotonia, cardiac conduction defects, diabetes, gastrointestinal malfunction, and central nervous system impairment⁴⁻⁷. The early disabilities are largely from distal muscle atrophy, leading to difficulty with performing tasks requiring fine dexterity, while the main causes of morbidity and mortality in late stage are from respiratory failure due to progressive muscle wasting. The congenital form has a high rate of neonatal mortality^{8,9}. Those who survive through infancy often succumb to respiratory failure in their forties⁹. DM1 is caused by CTG nucleotide repeat expansion within the 3′ Untranslated Region (3′-UTR) of the Dystrophia Myotonica Protein Kinase (DMPK) gene (10). The expanded CTG repeats encode toxic CUG RNAs that causes disease largely through RNA gain-of-function^{1,7,11-13}.

Ongoing therapeutic strategies primarily target the degradation of the expanded mutant transcripts¹⁴⁻²⁰. The most promising approach currently under development is ASOs. ASO therapy has demonstrated favorable efficacy in the pre-clinical studies^{19,21}. However, the initial clinical trial has to be on hold due to no noticeable therapeutic effect. It is conceivable that the mutant transcript knockdown is not permanent, making these strategies challenging for long-term therapy. The endogenous myogenesis or muscle regeneration is defective in DM1²²⁻³⁰. Thus, exogenous cell transplantation is a viable option. Cell transplantation for muscular dystrophy was previously tested on Duchenne Muscular Dystrophy (DMD). However, the results were disappointing. The main issue was the source of the transplanted cells. All early studies used allogenic myoblasts derived from muscle biopsy tissues. The initial immune reaction killed 75-80% of the transplanted cells³¹⁻³⁷.

Moreover, myoblasts have their own intrinsic defects when it comes to cell-based therapy. Traditionally, myoblasts are acquired from in vitro culture of isolated satellite cells from the muscle tissues. These myoblasts can only proliferate for certain passages and further ex vivo expansion degrades their myogenic capacity³⁸. Upon transplantation, surviving myoblasts have shown poor migration and fail to replenish the satellite compartment, making it impossible to maintain a sustained effect^{38,39}. It is thus understandable that myoblast transplantation for DM1 has never been tested in clinical trials. Other human muscle stem cells have been investigated for cell-based therapy⁴⁰⁻⁴⁶, but they all rely on isolation from live human muscle tissues. Large quantities of cells are needed for autologous cell transplantation therapy. However, due to the very nature of DM1 disease progression, it is almost impossible to manufacture a therapeutic quantity of muscle stem cells from DM1 patients' muscle tissue without causing severe, permanent damage to already-atrophied muscle. Moreover, satellite cells from DM1 patients are defective in muscle regeneration^{22-30,47}.

Accordingly new therapeutic and curative methodologies are desperately needed.

### SUMMARY

In general, the present disclosure provides methods and mechanisms for treating and/or alleviating myotonic dystrophy type 1 (DM1). According to a first embodiment, the present disclosure provides methods and mechanisms for editing the DMPK gene. According to a further embodiment, the present disclosure provides a mechanism for editing the genome of DM1 cells. According to another embodiment, the present disclosure provides genome-edited cells. According to a still further embodiment, the present disclosure provides a method of treating patients with DM1. According to yet another embodiment, the method of treatment comprises implanting skeletal myogenic progenitor cells (SMPCs) derived from genome-edited human-derived DM1 induced pluripotent stem cells (iPSCs) into those patients. According to another embodiment, the method of treatment comprises in vivo genome editing of the patient with DM1.

### BRIEF DESCRIPTION OF THE DRAWINGS

FIG. 1 is a schematic illustration of targeted insertion of an exemplary insertion cassette containing exemplary PASs flanked by homologous arms into a DM1 DMPK gene

FIG. 2 is a schematic illustration of the genome-editing process and the expected results.

FIG. 3 is a first exemplary AAV vector map according to an embodiment of the present disclosure.

FIG. 4 is a second exemplary AAV vector map according to another embodiment of the present disclosure.

FIG. 5 is a third exemplary AAV vector map according to still another embodiment of the present disclosure.

FIG. 6 is a flowchart illustrating an exemplary method for treating a patient according to an embodiment of the present disclosure where the patient's own cells are used to produce iPSC cells which are then genome edited and then differentiated, and wherein the differentiated cells are then implanted into the patient.

FIG. 7 is an image of genome edited iPSCs carrying the DM1 mutation.

FIG. 8 is an image of unedited iPSCs carrying the DM1 mutation.

FIG. 9 is an agarose gel image showing the results of genotyping by junctional PCR and TP-PCR showing the correct insertion of the PolyA cassettes in the 3′ UTR in genome-edited cells produced using the methods disclosed herein.

FIG. 10 is an agarose gel image showing the results of RT-PCR showing the expression of normal DMPK transcripts in a genome-edited clone produced using the method described herein.

FIG. 11 is the results of a RT-PCR of cytoplasmic DMPK RNA showing significantly increased cytoplasmic DMPK RNA in NSCs derived from genome-edited J-6 iPSCs compared to parental DM-03 derived NSCs. *p<0.01 by Student's t test.

FIG. 12 is a schematic view of the primer positions used for experiments described herein.

FIG. 13 is an agarose gel image showing the reversal of aberrant splicing patterns in cardiac troponin T (CTNT), insulin receptor (INSR), and muscleblind-like 2 (MBNL2) in cardiomyocytes derived from genome-edited iPSC (J-6) according to the methods of the present disclosure.

FIG. 14 is the results of quantitative analysis showing the reversal of aberrant splicing patterns in CTNT.

FIG. 15 is the results of quantitative analysis showing the reversal of aberrant splicing patterns in INSR.

FIG. 16 is the results of quantitative analysis showing the reversal of aberrant splicing patterns in MBNL2.

FIG. 17 is an agarose gel image showing the reversal of aberrant splicing patterns in microtubule-associated protein tau (MAPT) and MBNL1, 2 in NSCs derived from genome-edited iPSC (J-6) according to the methods of the present disclosure.

FIG. 18 is the results of quantitative analysis showing the reversal of aberrant splicing patterns in MAPT.

FIG. 19 is the results of quantitative analysis showing the reversal of aberrant splicing patterns in MBNL1.

FIG. 20 is the results of quantitative analysis showing the reversal of aberrant splicing patterns in MBNL2.

### DETAILED DESCRIPTION

In general, the present disclosure provides methods and mechanisms for treating and/or alleviating myotonic dystrophy type I (DM1). According to a first embodiment, the present disclosure provides methods and mechanisms for editing the genome of DM1 cells. For the purposes of the present disclosure, the terms “edited” or “corrected” (as in a “edited genome,” “edited gene,” “edited cell,” “corrected genome,” etc.) and their variants (“editing,” “correcting,” etc.) are used to refer to a genome, gene, cell, etc. that has been altered (or which has been derived from a genome, gene, cell etc. that has been altered) to reduce, reverse, or eliminate the DM1 phenotype. It is noted that in the context of the present disclosure, the terms do not necessarily require the removal or direct alteration of the mutation per se, but rather eliminates the toxic products from the mutation by modifying the genome adjacent to the mutation.

DM1 is caused by CTG nucleotide repeat expansion within the 3′ Untranslated Region (3′-UTR) of the Dystrophia Myotonica Protein Kinase (DMPK) gene (a). The DMPK gene as well as the DM1 mutation is described in, for example, Fu et al. (1992) An unstable triplet repeat in a gene related to myotonic muscular dystrophy. Science 255 (5049): 1256-1258.

The expanded CTG repeats encode toxic CUG RNAs that cause disease largely through RNA gain-of-function. The present disclosure provides a strategy for eliminating the expanded CUG mutant transcripts by preventing transcription of the CUG repeats. According to an embodiment, CUG mutant transcripts are eliminated by insertion of one or more polyadenylation signals (PASs) between the stop codon and expanded CTG repeats of the DMPK gene. For the purposes of the present disclosure, polyadenylation signals are defined as a DNA sequence that signal the post-transcription procession of messenger RNA (mRNA) for adding adenine bases. PASs that are suitable for use in the present disclosure include both naturally occurring and synthetic polyadenylation signals, including, but not necessarily limited to, the specific PASs identified throughout the present disclosure. According to a first embodiment, the PASs are inserted into the 3′-UTR of the DMPK gene.

Numerous genome editing techniques have been developed and several are becoming increasingly well-known for their efficacy and utility in both in vitro and in vivo applications. Exemplary genome editing techniques typically rely on engineered nucleases such as meganucleases, zinc finger nucleases (ZFNs), transcription activator-like effector-base nucleases (TALENs) and the clustered regularly interspaced short palindromic repeats (CRISPR/Cas9) system to insert “donor” genetic material, typically in the form of an “insertion cassette” into a specific location of a “recipient” genome. Accordingly, those of skill in the art will understand that any of the above-identified systems could be adapted to insert the PASs between the stop codon and expanded CTG repeats of the DMPK gene.

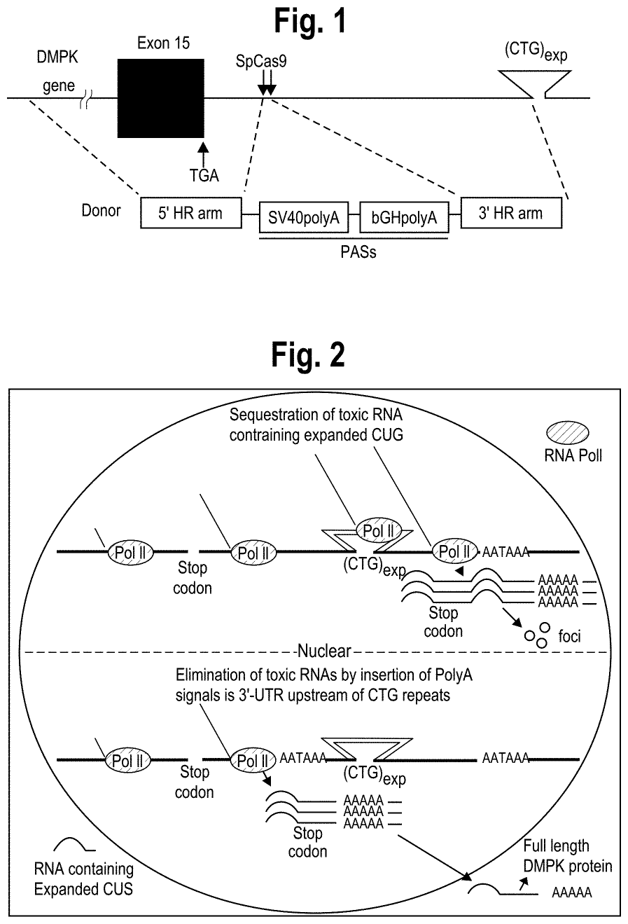

FIG. 1 is a schematic illustration of targeted insertion of an exemplary insertion cassette containing exemplary PASs flanked by homologous arms into a DM1 DMPK gene. In general, the homologous arms are DNA sequences that are homologous to respective 5′ and 3′ regions of the DMPK gene that flank the desired insertion site. As described in greater detail below, the insertion cassette could be further altered to include additional sequences that could provide other functionalities, abilities, or characteristics that could, for example, contribute to the delivery, efficacy, safety, and/or function of the cassette, edited gene, environment, process and/or therapeutic purpose. FIG. 2 is a schematic illustration of the genome-editing process and the expected results. As shown, insertion of the PASs results in elimination of the toxic RNAs from the CTG repeats and expression of the full length DMPK protein.

Accordingly, for descriptive purposes, the present disclosure provides a specific example wherein a CRISPR/Cas9 system is used to insert several PASs into a DM1 DMPK gene. According to a specific embodiment, the insertion cassette is inserted into an insertion site in the 3′-UTR of the DMPK gene via a homology directed repair (HDR) triggered by a double strand break (DSB) that is created by a site-specific guide RNA (gRNA)-CRISPR/Cas9 Those of skill in the art will understand that the insertion site is typically determined by a pair of gRNA and SpCas9 nickase being used. According to a first specific example, the Streptococcus pyogenes Cas9 (SpCas9) nickase system may be used as the mechanism for inserting the PASs. Additional information regarding the SpCas9 nickase system may be found, for example, in Cong, et al. (2013) Multiplex genome engineering using CRISPR/Cas systems. Science 339, 819-823; Ran et al., (2013). Double nicking by RNA-guided CRISPR Cas9 for enhanced genome editing specificity. Cell 154, 1380-1389; Cho et al., (2014). Analysis of off-target effects of CRISPR/Cas-derived RNA-guided endonucleases and nickases. Genome Res. 24, 132-141; and Jinek et al., (2012). A programmable dual-RNA-guided DNA endonuclease in adaptive bacterial immunity. Science 337, 816-821.

In general, the SpCas9 D10A nickase is created by an aspartate-to-alanine substitution (D10A) in the RuvC I domain of SpCas9 that can produce a nick guided to a specific genome site using a pair of sequence-specific gRNAs. The single nick in the genome is typically repaired either seamlessly via the single strand break repair pathway or through high-fidelity HDR when an ectopic donor exits. However, when there is an adjacent nick on the opposite strand from the second gRNA-SpCas9 nickase, it can cause double-strand breaks. These double-strand breaks are repaired preferentially by HDR when a donor exists, which allows for the insertion of ectopic DNAs, such as the insertion cassette described above. The double-strand break is relatively more specific due to the requirement of more than double the length of DNA recognition sequence compared to the single wild-type SpCas9. The present disclosure provides a pair of gRNAs (Sp870/Sp870U)-SpCas9 nickase that facilitate the insertion of PolyA signals in the 3′-UTR upstream of the CTG repeats. In general, targeting efficiency may be increased by increasing the total length of homologous arms. However, it is suggested that the homologous arms include between 100 and 5000 basepairs (bp). According to a specific embodiment, homologous arms having 281 bp (5′ arm 97 bp, 3′arm 184 bp) were found to be sufficient to induce HDR.

Of course, it should be noted that while the present disclosure and Examples may identify specific PASs, homologous arm sequences, and nickase systems, those of skill in the art will be familiar with or understand that variants, variations, and/or substitutions of these or other PASs, homologous arm sequences, and/or nuclease (for example ZFN, TALEN, SaCas9, cpf1, ScCas9, dCas9-Fokl, SpCas9-HF1, eSpCas9, HypaCas9, etc) systems are similarly suitable, and are thus considered to be within the scope of this disclosure.

According to a further embodiment, the present disclosure provides for the generation of viable genome edited cells. According to this method, DM1 cells (i.e. cells exhibiting the DM1 phenotype due to the presence of a CTG nucleotide repeat expansion in the DMPK gene) undergo genome editing wherein PASs are inserted into the DMPK gene to prevent the transcription of the mutant CTG repeats. According to an embodiment, the PASs may be inserted using a vector system. Suitable commercially available vector systems include, but are not limited to, Recombinant Adeno-associated virus (AAV) vector systems.

AAV vectors have been well-accepted in gene therapy for human diseases and efficiently transduce many cell types both in vivo and ex vivo. Methods for transducing cell types with AAV vectors are described in Khan et al. Engineering of human pluripotent stem cells by AAV-mediated gene targeting. Mol Ther. 2010; 18:1192-1199; Russell et al. Human gene targeting by viral vectors. Nat Genet. 1998; 18:325-330; Chamberlain et al., Gene targeting in stem cells from individuals with osteogenesis imperfecta. Science. 2004; 303:1198-1201; and Agrawal et al., Generation of recombinant skin in vitro by adeno-associated virus type 2 vector transduction. Tissue Eng. 2004; 10:1707-1715.

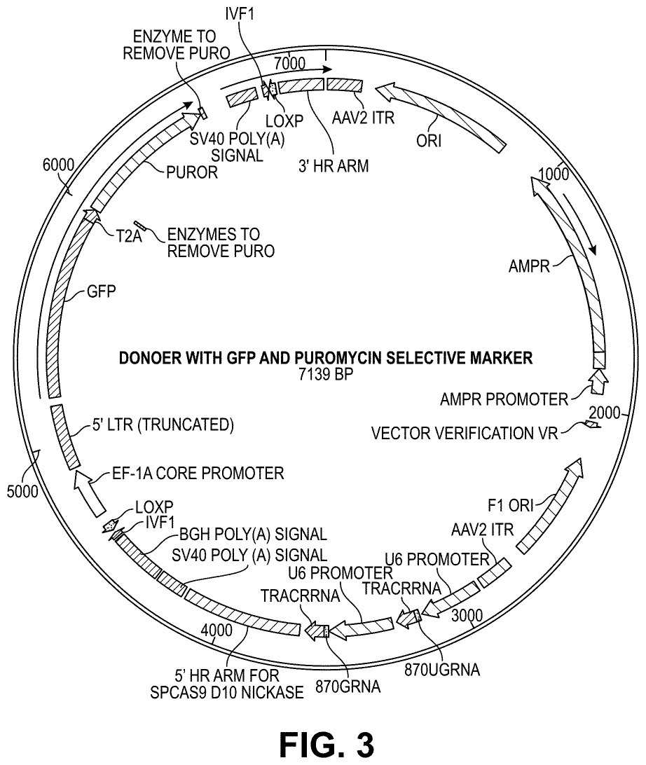

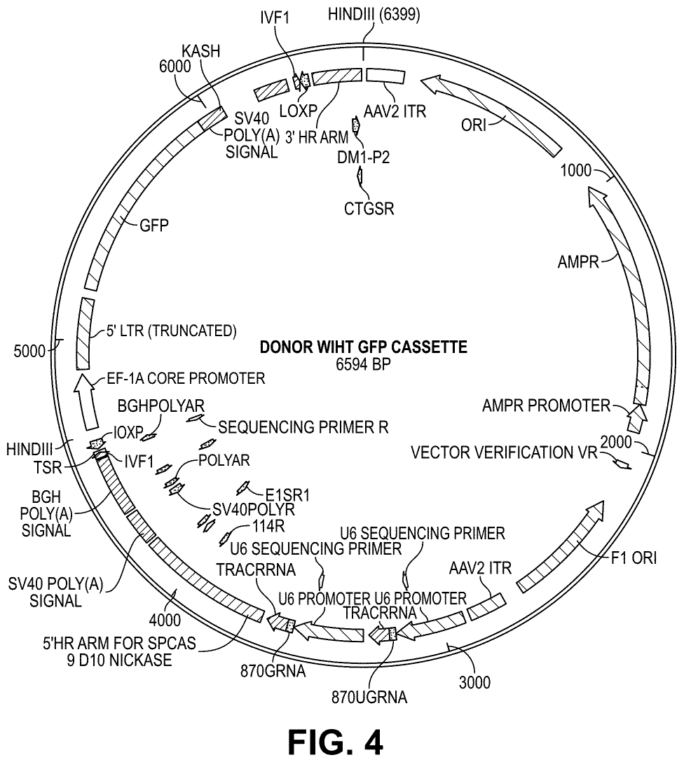

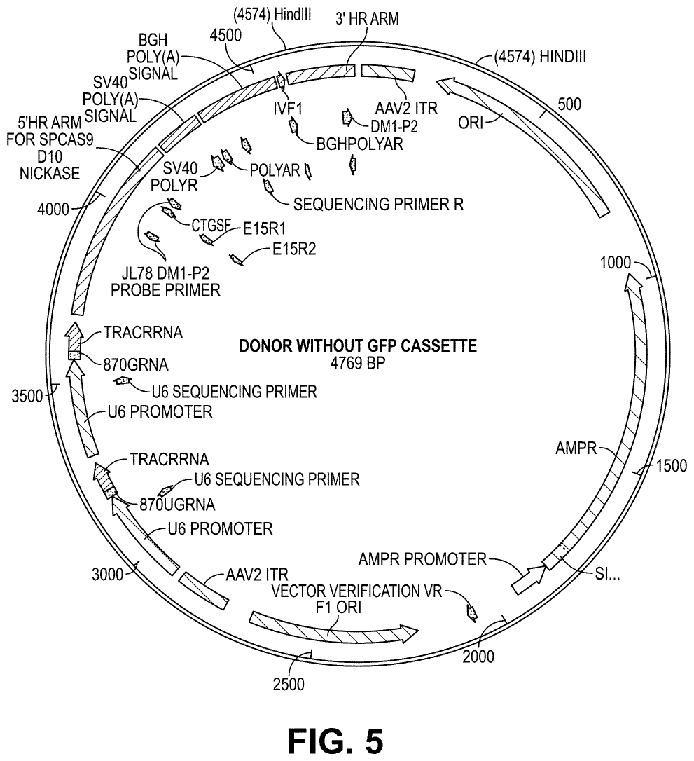

Exemplary AAV vector maps according to embodiments of the present disclosure are shown in FIGS. 3-5, In FIG. 3, an exemplary AAV-based donor vector includes AAV2 ITR regions flanking the transgene. The two U6gRNA sequences, homologous arms and PolyA signal, and any desired selective markers (e.g., GFP, Puromycin, etc.) are engineered between the two ITRs. Of course it will be understood that other embodiments could include other, additional, or no selective markers. As shown in FIG. 4, the puromycin selective marker shown in can be removed by the BsiWI restriction enzyme without affecting the GFP, which may be useful for tracking purposes in, for example, an in vivo application. Moreover, according to some embodiments, the pair of gRNAs may be included in the vector carrying the PolyA cassette, but not in the SpCas9 cassette. The separation of gRNAs and Cas9 may avoid idle DSBs when no donor exists. This will further decrease off-target cleavage and increase the rate of desired DSBs/integration, e.g. if there is no donor, there will not be any DSBs. Other possibly desirable enhancements might include, for example, optimization of the promotor region, or alterations to the gRNA to reduce off-target mutations (e.g., the addition of two extra guanine nucleotides to the 5′ end or using truncated gRNAs). As shown in FIG. 5, for clinical applications, the GFP-expressing cassette can also be removed by Hind III to avoid unnecessary ectopic DNAs.

Those of skill in the art will understand that promoter selection can be an important factor in determining gene therapy efficiency. However, it should be noted that therapeutic genome editing is different from gene therapy in which sustained strong expression is preferred. For example, only transient expression of Cas9 is required for therapeutic genome editing. According to some embodiments, it may be desirable to avoid sustained strong expression which may, for example, have detrimental effects on the cells due to potential dose-dependent off-target effects. As stated above, in some embodiments it may be desirable to use a promotor which is specific to the biological system to which the gene therapy is being directed (i.e. a muscle specific promotor.) However, for multisystem diseases such as DM1, it may be preferable to select constitutive promoters (EF-1alpha, CMV, human U6, H1, etc.).

For the expression of SpCas9 nickase, it may be desirable to use a smaller promoter, such as the minimal CMV promoter (180 bp) and minimal synthetic polyadenylation signal because of the packaging size limitation of the AAV vector and the large size of the SpCas9 (4101 bp). Suitable minimal CMV promotors are described in, for example, Senis E, et al. CRISPR/Cas9-mediated genome engineering: an adeno-associated viral (AAV) vector toolbox. Biotechnol J. 2014; 9:1402-1412; and Schmidt et al., CRISPR genome engineering and viral gene delivery: a case of mutual attraction. Biotechnol J. 2015; 10:258-272. Suitable minimal synthetic polyadenylation signals are described, for example, in Swiech et al. In vivo interrogation of gene function in the mammalian brain using CRISPR-Cas9. Nat Biotechnol. 2015; 33:102-106; et al. CRISPR/Cas9-mediated genome engineering: an adeno-associated viral (AAV) vector toolbox. Biotechnol J. 2014; 9:1402-1412; Gray et al. Optimizing promoters for recombinant adeno-associated virus-mediated gene expression in the peripheral and central nervous system using self-complementary vectors. Hum Gene Ther. 2011; 22:1143-1153; and Levitt et al. Definition of an efficient synthetic poly(A) site. Genes Dev. 1989; 3:1019-1025.

One limitation of AAV vectors is their small packaging capacity that is generally considered to be <5 kb, though up to 6 kb has been reported. Accordingly, one embodiment provides for split AAV/SpCas9 cassettes that reconstitute/dimerize by intein (a peptide similar to an intron in the genome), chemical, or sgRNA to form a functional unit after delivery. Double nicking using split-SpCas9 in an AAV system is described, for example, in Truong, et al. Development of an intein-mediated split-Cas9 system for gene therapy. Nucleic Acids Res. 2015; 43:6450-6458.

According to various embodiments, the genome edited cells may be myogenic cells or cells able to differentiate into myogenic cells. According to a specific embodiment, the genome edited cells may be induced pluripotent stem cells (iPSCs), mesenchymal stem cells, or engineered somatic cells that can be differentiated into skeletal muscle cells. According to another specific embodiment, the genome edited cells may also be bone marrow hematopoietic stem cells, T cells, B cells, monocytes, and macrophages.

iPSC cells are similar to embryonic stem cells (ESC) in that iPSCs can be expanded indefinitely at the pluripotent stage and are able to differentiate into all three primary germ layers and, therefore, potentially into all the cell types of the body. The advantage of iPSC is the prospect of generating unlimited quantities of specific cell population for regenerative purposes. iPSCs are derived from somatic cells and the process does not involve the use of embryonic cells, removing ethnical concerns.

It will be noted that if the targeted integration of PAS relies on HDR, as in the SpCas9 system described above, it may be desirable to target cells during their early stage, when there are ample stem cell pools, because HDR is more likely in S and G2 phases during a cell cycle. However, it will be understood that different systems will have different optimal conditions and timing and such factors can and should be selected as needed.

According to various embodiments, the genome edited cells may be from or derived from cells from an individual who has DM1. Any suitable method of generating iPSCs can be used. Examples of suitable method for generating iPSCs include direct reprogramming of human somatic cells using retrovirus such as those described in Takahashi et al. (2007) Induction of pluripotent stem cells from adult human fibroblasts by defined factors. Cell 131,861-872; Yu et al. (2007) Induced pluripotent stem cell lines derived from human somatic cells. Science 318,1917-1920; Kastenberg et al. (2008) Alternative sources of pluripotency: science, ethics, and stem cells. Transplant Rev (Orlando) 22,215-222; and Xia et al. (2013). Generation of neural cells from DM1 induced pluripotent stem cells as cellular model for the study of central nervous system neuropathogenesis. Cell Reprogram 15: 166-177. Other suitable methods for obtaining iPSC cells also include the integration-free method described in Zhou et al. Integration-free methods for generating induced pluripotent stem cells. Genomics Proteomics Bioinformatics. 2013 October; 11(5):284-7. doi: 10.1016/j.gpb.2013.09.008. Of particular note, iPSC cells can be derived from patient samples that are easily and even non-invasively obtained like skin, saliva, blood, or urine samples. Once the iPSCs having the DM1 mutation are obtained, the cells can be edited using the above described method of inserting a PAS sequence into the DMPK gene.

According to a still further embodiment, the genome edited iPSCs may then be differentiated into genome-edited skeletal myogenic progenitor cells (SMPCs). Skeletal muscle has strong potential to regenerate itself upon damage, an effect mainly contributed by adult satellite cells. In adult muscle, satellite cells are found in very small number between the basement membrane and the sarcolemma of muscle fibers. They are quiescent and express PAX7 at normal levels. Upon activation by muscle damage, they can self-renew to maintain the stem cell pool. Satellites cells can undergo symmetric division, which gives rise to two identical daughter satellite cells, and asymmetric division, which gives rise to one daughter satellite cell and one cell committed to myoblast that further proliferate, differentiate, fuse, and lead to new myofiber formation and reconstitution of a functional contractile apparatus. Recently, multiple protocols have been developed to differentiate human iPSCs into myogenic lineage cells. Examples are provided, for example, in Maffioletti et al. Efficient derivation and inducible differentiation of expandable skeletal myogenic cells from human ES and patient-specific iPS cells. Nat Protoc. 2015; 10:941-958; Chal et al. Generation of human muscle fibers and satellite-like cells from human pluripotent stem cells in vitro. Nat Protoc. 2016; 11:1833-1850; Darabiet al., Derivation of Skeletal Myogenic Precursors from Human Pluripotent Stem Cells Using Conditional Expression of PAX7. Methods Mol Biol. 2016; 1357:423-439; Hosoyama et al. Derivation of myogenic progenitors directly from human pluripotent stem cells using a sphere-based culture. Stem Cells Transl Med. 2014; 3:564-574; Chal et al. Differentiation of pluripotent stem cells to muscle fiber to model Duchenne muscular dystrophy. Nat Biotechnol. 2015; 33:962-969; and Swartz et al. A Novel Protocol for Directed Differentiation of C9orf72-Associated Human Induced Pluripotent Stem Cells Into Contractile Skeletal Myotubes. Stem Cells Transl Med. 2016; 5:1461-1472. These cells are able to fuse to host myofibers and exhibit superior strength. They can also seed the muscle satellite cell compartment. This is particularly important as continuous cycles of myofiber degeneration and regeneration in advanced degenerative muscular dystrophy may exhaust the satellite cell reserves and as such lose their regenerative capacity. Restoration of the satellite cells pool will restore the regenerative capacity of the muscle and maintains sustained effects.

According to a specific embodiment, genome edited iPSCs are cultured using suitable culturing conditions. For example, iPSCs can be maintained using protocols such as those disclosed in Gao Y, Guo X, Santostefano K et al. Genome Therapy of Myotonic Dystrophy Type 1 iPS Cells for Development of Autologous Stem Cell Therapy. Mol Ther. 2016; 24:1378-1387; Xia G, Gao Y, Jin S et al. Genome modification leads to phenotype reversal in human myotonic dystrophy type 1 induced pluripotent stem cell-derived neural stem cells. Stem Cells. 2015; 33:1829-1838; Xia G, Santostefano K, Hamazaki T et al. Generation of human-induced pluripotent stem cells to model spinocerebellar ataxia type 2 in vitro. J Mol Neurosci. 2013; 51:237-248; and Xia G, Santostefano K E, Goodwin M et al. Generation of neural cells from DM1 induced pluripotent stem cells as cellular model for the study of central nervous system neuropathogenesis. Cell Reprogram. 2013; 15:166-177. According to some embodiments, these protocols may be modified to meet the criteria of clinically-clean iPSCs, including the use of feeder-free, xeno-free culture and coating media. While common cultures call for the use of an extracellular matrix such as, for example, the Corning Matrigel matrix (Corning, N.Y., N.Y.), it should be noted that the Corning Matrigel matrix contains a mixture of matrix proteins and growth factors of non-human origin. Accordingly, for applications wherein the cells are ultimately to be implanted in a human subject, it may be desirable to use cultures conditions that do not utilize non-human origin additives. According to a specific example, cultured cells may be coated with laminin and collagen IV from human cell culture (for example, Sigma-Aldrich C6745, Sigma-Aldrich Co.) and adapted to Laminin 521 coating culture conditions. Laminin 521 (LaminStem™ 521,05-753-1F, Biological Industries) is a chemically defined, animal component-free, xeno-free matrix. Those of skill in the art will be familiar with other suitable culturing conditions as well as the adaptation of those conditions for the specific uses of the presently described genome edited cells.

Differentiation of the edited iPSCs into edited SMPCs can be achieved using methods described in, for example, Maffioletti S M, Gerli M F, Ragazzi M et al. Efficient derivation and inducible differentiation of expandable skeletal myogenic cells from human ES and patient-specific iPS cells. Nat Protoc. 2015; 10:941-958; Chal J, Al Tanoury Z, Hestin M et al. Generation of human muscle fibers and satellite-like cells from human pluripotent stem cells in vitro. Nat Protoc. 2016; 11:1833-1850; Darabi R, Perlingeiro R C. Derivation of Skeletal Myogenic Precursors from Human Pluripotent Stem Cells Using Conditional Expression of PAX7. Methods Mol Biol. 2016; 1357:423-439 Hosoyama T, McGivern J V, Van Dyke J M et al. Derivation of myogenic progenitors directly from human pluripotent stem cells using a sphere-based culture. Stem Cells Transl Med. 2014; 3:564-574; Chal J, Oginuma M, Al Tanoury Z et al. Differentiation of pluripotent stem cells to muscle fiber to model Duchenne muscular dystrophy. Nat Biotechnol. 2015; 33:962-969; and/or Swartz E W, Baek J, Pribadi M et al. A Novel Protocol for Directed Differentiation of C9orf72-Associated Human Induced Pluripotent Stem Cells Into Contractile Skeletal Myotubes. Stem Cells Transl Med. 2016; 5:1461-1472. An exemplary method of induction is described, for example, in Chal et al, incorporated above. Briefly, cells are plated into 6-well plates coated with Laminin 521 and collagen IV at a density of 3×10⁴ per cm². Differentiation is initiated by activation of the WNT signaling pathway using CHIR99021 (Tocris Bioscience) and inhibition of the BMP signaling pathway using LDN193189 (Stemgent Inc.). For the following days, FGF, IGF, HGF are added sequentially. On day twenty, the immature SMPCs are split (at a density of 70,000 cells/cm2), isolated and expanded. Of course those of skill in the art will be familiar with other suitable protocols.

According to a still further embodiment, the present disclosure provides methods and mechanisms for implanting genome edited cells into a subject. Normal satellite cells can be isolated from muscle tissues and cultured in vitro, but simple expansion of satellite cells in culture results in rapid differentiation and loss of their regenerative properties unless they are transplanted directly after isolation or along with the entire myofiber. Transplantation of these satellite cells along with human muscle fiber fragments into irradiated muscle of immunodeficient mice resulted in robust engraftment, muscle regeneration and proper homing of human PAX7′ satellite cells to the stem cell niche. However, as discussed above, in DM1 subjects with profound muscle atrophy, it may be impossible to isolate and expand enough satellite cells from biopsy muscles for cell transplantation.

Accordingly, it may be desirable to utilize iPSCs, as the capability of iPSCs to generate an unlimited source of SMPCs overcomes the cell number hurdle. Moreover, or personalized cell-based therapy, SMPCs derived from patient-specific iPSCs have theoretically no risk of rejection. The prerequisite is to acquire genome-edited SMPCs. Genome-edited SMPCs will generate new or fuse with existing myofibers and eventually substitute all the diseased myofibers through repetitive cycles of degeneration and regeneration. The replacement of diseased muscle fibers by genome-edited muscle fibers may offer a permanent cure for the local muscle pathology.

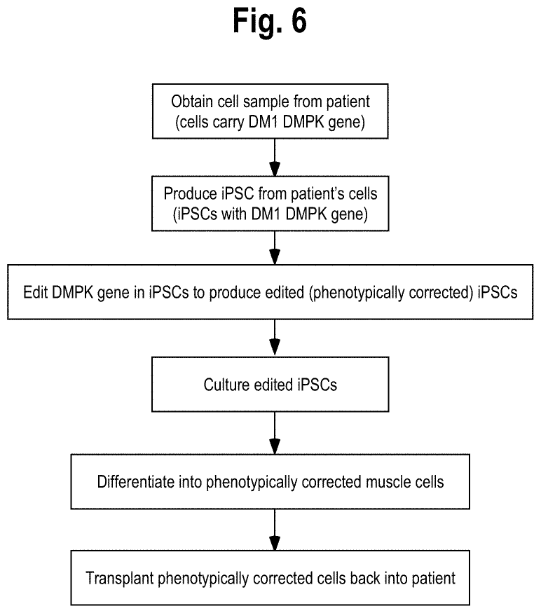

Accordingly, the subject into which the genome edited cells are implanted may, for example, be suffering from DM1. The subject may, for example, be a human who has been diagnosed with DM1. According to an example of this embodiment, a cell sample from a subject who has been diagnosed with DM1(referred to herein as the “patient”) may be obtained using any reasonable method, including, but not necessarily limited to, those described above. The cell sample may then be used to produce patient-specific iPSCs, containing the DM1 mutation. The genomes of the DM1 iPSCs can then be edited using the methods described above. The genome-edited iPSCs can then be differentiated into myogenic lineage cells to produce genome-edited SMPCs. The genome-edited SMPCs can then be transplanted into the patient where the cells satellite to fuse to host myofibers. They can also seed the muscle satellite cell compartment. This is particularly important as continuous cycles of myofiber degeneration and regeneration in advanced degenerative muscular dystrophy may exhaust the satellite cell reserves and as such, lose their regenerative capacity. Restoration of the satellite cells pool will restore the regenerative capacity of the muscle and maintains sustained effects. Transplantation of the genome-edited SMPCS may be performed using the methods described in (Law P K, Bertorini T E, Goodwin T G et al. Dystrophin production induced by myoblast transfer therapy in Duchenne muscular dystrophy. Lancet. 1990; 336:114-115, or Gussoni E, Pavlath G K, Lanctot A M et al. Normal dystrophin transcripts detected in Duchenne muscular dystrophy patients after myoblast transplantation. Nature. 1992; 356:435-438) This embodiment is illustrated in the flowchart shown in FIG. 6.

According to a first exemplary embodiment, 1×10⁴ to 3×10⁵ SMPCs can be injected directed into each site of the muscle. According to other embodiments, multiple sites could be injected based on the volume of the muscle.

According to a still further embodiment, the present disclosure provides a method for in vivo editing of a living subject's DM1 cells. Various methods of genome editing are identified above and those of skill in the art will understand that the specific methods described herein could be tailored or altered to utilize or incorporate various other gene editing methods.

Again, for the purposes of illustration, the present disclosure provides a specific example wherein a CRISPR/Cas9 system can be used to insert several PASs into an in vivo DM1 DMPK gene. In this embodiment, an AAV vector packaged, for example, into AAV capsid to generate infection virions or a non-viral vector delivers SpCas9 and a gene editing insertion cassette containing the gRNA, the PASs, and the homologous arms to the genomes of DM1 cells within a living subject, enabling editing of the subject's own cells, effectively acting as a cure for the DM1 phenotype. Of course the AAV-based donor vector is just one example of a suitable delivery system and other suitable delivery mechanisms are contemplated by and considered to be within the scope of the present disclosure. Examples of other suitable delivery mechanisms include, but are not limited to, electroporation of Cas9/gRNA Ribonucleoprotein (RNP), nanoparticles, and lipid-based transfection. Importantly, DM1 is known to be inherited and can present as congenital myotonic dystrophy type 1. Accordingly, it is possible to identify patients based on family history and genetic testing even if they are asymptomatic. In fact, the presently described in vivo methodology could even be applied during early childhood, neonatal stage, or even in utero, offering the best chance to reduce, halt, or even prevent the onset or progression of the disease.

According to yet another embodiment, the present disclosure provides a method for promoting an environment that stimulates transplantation, growth, generation and regeneration of the edited DM1 cells. Specifically, the present disclosure contemplates the delivery of trophic (and possibly other helpful) factors to edited and transplanted DM1 cells via the administration of engineered monocyte cells.

Early studies suggest that although satellite cells can proliferate and form myotubes and myofibers in vitro, their regeneration potential in vivo might be largely determined by host stem cell niche and microenvironment. Following injury of adult muscle, resident satellite cells, which are mostly quiescent, re-enter the cell cycle and generate myoblasts that will participate in myofiber reconstitution or repair. The efficient reconstitution of functional muscle requires the coordinated action of other cell types including macrophages, fibro-adipogenic precursors, interstitial connective tissue and endothelial cells for blood vessel formation. Macrophage are particularly important. For stem cell therapy, scar tissue inevitably forms a barrier to repopulation by implanted cells. Maintaining a balance between inflammation and subsequent connective remodeling is of particular relevance to the treatment of muscular dystrophy. Improving the host environment is therefore a critical component of cell transplantation therapies.

The presence of monocyte/macrophages is mandatory for skeletal muscle regeneration. Mice deficient in chemokine receptor or ligand show impaired muscle regeneration, which is associated with a dramatic decrease in macrophage infiltration into the muscle and was reversed by wild type bone marrow transplantation. Depletion of circulating monocytes at the time of muscle injury totally prevents muscle regeneration. Patrolling monocytes selectively traffic to the sites of muscle degeneration/inflammation and differentiate into macrophages. Initially, these macrophages present as pro-inflammatory macrophage (MI) that will clear muscle debris and stimulate myogenic cell proliferation. Then, the phagocytosis of muscle debris induce a switch of pro-inflammatory M Itoward anti-inflammatory macrophages (M2), which proliferate and promote muscle differentiation. Macrophages also improve survival, proliferation and migration of engrafted SMPCs. In DM1 muscle, there is infiltration of monocytes/macrophages. Accordingly, it is reasonable to believe that the unique degenerative/inflammatory environment will attract monocytes, which opens a route to bring in trophic factor through systemic administration of engineered monocytes.

One of the important trophic factors is Insulin-like growth factor 1 (IGF-1). IGF-1 has been implicated as central regulator of muscle regeneration. It is an important factor in the current in vitro SMPC differentiation protocol. IGF-1 accelerates muscle regeneration and restores muscle function and architecture by prolonging the regenerative potential of skeletal muscle through increasing satellite cell activity, recruiting circulating stem cells at sites of muscle degeneration, modulating inflammatory factors, reducing muscle necrosis and fibrosis, and elevating signaling pathways associated with muscle survival and regeneration. The beneficial effects of local expression of IGF-1 on muscle regeneration was shown in degenerative processes such as muscular dystrophy and Amyotrophic Lateral Sclerosis and even in sarcopenia related to aging.

Thus according to an embodiment, the present disclosure provides local delivery of IGF-1 by genetically-altered IGF-1 producing monocytes. The advancement of iPSC technology and therapeutic genome editing allows us to generate IGF-1 producing monocytes from iPSC in large quantities. Protocols for large scale production of monocytes for cell transplantation are described, for example, in Lachmann et al. Large-scale hematopoietic differentiation of human induced pluripotent stem cells provides granulocytes or macrophages for cell replacement therapies. Stem Cell Reports. 2015; 4:282-296; Yanagimachi et al. Robust and highly-efficient differentiation of functional monocytic cells from human pluripotent stem cells under serum- and feeder cell-free conditions. PLoS One. 2013; 8:e59243; van Wilgenburg et al., Efficient, long term production of monocyte-derived macrophages from human pluripotent stem cells under partly-defined and fully-defined conditions. PLoS One. 2013; 8:e71098; and Karlsson et al. Homogeneous monocytes and macrophages from human embryonic stem cells following coculture-free differentiation in M-CSF and IL-3. Exp Hematol. 2008; 36:1167-1175.

Human iPSC-derived monocytes/macrophages resemble anti-inflammatory M2-polarized macrophages expressing classical macrophage markers (CD45, CD 14, and CD 163). These cells share ontogeny with MYB-independent tissue-resident macrophages, which will stay longer in the tissue than bone marrow hematopoietic stem cell-derived monocytes/macrophages. Accordingly, the iPSC-derived IGF-1 producing monocytes/macrophages should exert long term effects. Young monocytes isolated from 2-week-old mice enhanced clearance of beta-amyloid plaques in Alzheimer mouse model. iPSC-generated monocytes resemble monocytes from yolk sac during embryogenesis. The infusion and infiltration of these young monocytes into degenerating muscle tissue may enhance clearance of muscle debris and create a better environment for muscle regeneration.

According to an exemplary embodiment, genetically altered IGF-1 producing monocytes may be generated by targeted insertion of an IGF-1 gene cassette in the safe harbor locus (for example. AAVS1 locus) in human genome. As a specific example, iPSC colonies such as those described above, are detached and resuspended in embryoid body (EB) culture medium containing BMP-4(50 ng/ml), VEGF (50 ng/ml), FGF (10 ng/ml) and Y-27632 (10 μM) at a concentration of 1.25×105. 1001 μl is then seeded to into 96-well ultra-low adherence plate for EB formation. After four days of EB differentiation, EBs are transferred into six-well tissue-culture plate (8 EBs per well) and cultured in medium containing IL-3 (25-50 ng/ml) and M-CSF (50-100 ng/ml).

The IGF-1 monocytes can then be injected into the patient receiving genome edited DM1 cells (or into a patient who has received treatment of genome edited DM1 cells of their own, or a DM1 or other patient without any specific therapy) in order to deliver IGF-1 to those areas where muscle regeneration has occurred, is occurring, or will occur.

DM1 is a systemic disorder. Not only muscles are affected, other systems, including central nervous system (CNS), are also affected. Of course those of skill in the art will appreciate that monocytes that express other factors that might be beneficial for muscle or other tissues or organs (for example CNS) regeneration could be developed and delivered using similar methodologies and thus are contemplated by the present disclosure. Examples of suitable factors include, but are not limited to: brain-derived neurotrophic factor (BDNF), Glia cell-derive neurotrophic factor (GDNF). Additional information and disclosure may be found, for example, in applicant's co-pending PCT Application Serial No. PCT/US18/61481, entitled Genome Edited iPSC-Derived Monocytes Expressing Trophic Factors, filed Nov. 16, 2018, which claims priority to U.S. provisional application No. 62/587,530, filed Nov. 17, 2017, which is hereby incorporated by reference for all purposes.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

### REFERENCES

1. Romeo V. Myotonic Dystrophy Type 1 or Steinert's disease. Adv Exp Med Biol. 2012; 724:239-257
2. Harper P. Myotonic Dystrophy, 3rd ed. London: WB Saunders, 2001
3. Campbell C, Levin S, Siu V M et al. Congenital myotonic dystrophy: Canadian population-based surveillance study. J Pediatr. 2013; 163:120-125 e121-123
4. Udd B, Krahe R. The myotonic dystrophies: molecular, clinical, and therapeutic challenges. Lancet Neurol. 2012; 11:891-905
5. Thornton C A. Myotonic dystrophy. Neurol Clin. 2014; 32:705-719, viii
6. Turner C, Hilton-Jones D. Myotonic dystrophy: diagnosis, management and new therapies. Cuff Opin Neurol. 2014; 27:599-606
7. Ashizawa T, Sarkar P S. Myotonic dystrophy types 1 and 2. Handb Clin Neurol. 2011; 101:193-237
8. Echenne B, Rideau A, Roubertie A et al. Myotonic dystrophy type I in childhood Long-term evolution in patients surviving the neonatal period. Eur J Paediatr Neurol. 2008; 12:210-223
9. Reardon W, Newcombe R, Fenton I et al. The natural history of congenital myotonic dystrophy: mortality and long term clinical aspects. Arch Dis Child. 1993; 68:177-181
10. Fu Y H, Pizzuti A, Fenwick R G, Jr. et al. An unstable triplet repeat in a gene related to myotonic muscular dystrophy. Science. 1992; 255:1256-1258
11. Ranum L P, Cooper T A. RNA-mediated neuromuscular disorders. Annu Rev Neurosci. 2006; 29:259-277
12. Le J E, Cooper T A. Pathogenic mechanisms of myotonic dystrophy. Biochem Soc Trans. 2009; 37:1281-1286
13. Gomes-Pereira M, Cooper T A, Gourdon G. Myotonic dystrophy mouse models: towards rational therapy development. Trends Mol Med. 2011; 17:506-517
14. Wheeler T M, Sobczak K, Lueck J D et al. Reversal of RNA dominance by displacement of protein sequestered on triplet repeat RNA. Science. 2009; 325:336-339
15. Le J E, Bennett C F, Cooper T A. RNase H-mediated degradation of toxic RNA in myotonic dystrophy type 1. Proc Natl Acad Sci USA. 2012; 109:4221-4226
16. Langlois M A, Le N S, Rossi J J, Puymirat J. Hammerhead ribozyme-mediated destruction of nuclear foci in myotonic dystrophy myoblasts. Mol Ther. 2003; 7:670-680
17. Warf M B, Nakamori M, Matthys C M et al. Pentamidine reverses the splicing defects associated with myotonic dystrophy. Proc Natl Acad Sci USA. 2009; 106:18551-18556
18. Mulders S A, van den Broek W J, Wheeler T M et al. Triplet-repeat oligonucleotide-mediated reversal of RNA toxicity in myotonic dystrophy. Proc Natl Acad Sci USA. 2009; 106:13915-13920
19. Wheeler T M, Leger A J, Pandey S K et al. Targeting nuclear RNA for in vivo correction of myotonic dystrophy. Nature. 2012; 488:111-115
20. Parkesh R, Childs-Disney J L, Nakamori M et al. Design of a bioactive small molecule that targets the myotonic dystrophy type 1 RNA via an RNA motif-ligand database and chemical similarity searching. J Am Chem Soc. 2012; 134:4731-4742
21. Jauvin D, Chretien J, Pandey S K et al. Targeting DMPK with Antisense Oligonucleotide Improves Muscle Strength in Myotonic Dystrophy Type 1 Mice. Mol Ther Nucleic Acids. 2017; 7:465-474
22. Sarnat H B, Silbert S W. Maturational arrest of fetal muscle in neonatal myotonic dystrophy. A pathologic study of four cases. Arch Neurol. 1976; 33:466-474
23. Sahgal V, Bernes S, Sahgal S et al. Skeletal muscle in preterm infants with congenital myotonic dystrophy. Morphologic and histochemical study. J Neurol Sci. 1983; 59:47-55
24. Farkas-Bargeton E, Barbet J P, Dancea S et al. Immaturity of muscle fibers in the congenital form of myotonic dystrophy: its consequences and its origin. J Neurol Sci. 1988; 83:145-159
25. Furling D, Coiffier L, Mouly V et al. Defective satellite cells in congenital myotonic dystrophy. Hum Mol Genet. 2001; 10:2079-2087
26. Furling D, Lemieux D, Taneja K, Puymirat J. Decreased levels of myotonic dystrophy protein kinase (DMPK) and delayed differentiation in human myotonic dystrophy myoblasts. Neuromuscul Disord. 2001; 11:728-735
27. Furling D, Doucet G, Langlois M A et al. Viral vector producing antisense RNA restores myotonic dystrophy myoblast functions. Gene Ther. 2003; 10:795-802
28. Thornell L E, Lindstom M, Renault V et al. Satellite cell dysfunction contributes to the progressive muscle atrophy in myotonic dystrophy type 1. Neuropathol Appl Neurobiol.2009; 35:603-613
29. Timchenko N A, Iakova P, Cai Z J et al. Molecular basis for impaired muscle differentiation in myotonic dystrophy. Mol Cell Biol. 2001; 21:6927-6938
30. Loro E, Rinaldi F, Malena A et al. Normal myogenesis and increased apoptosis in myotonic dystrophy type-1 muscle cells. Cell Death Differ. 2010; 17:1315-1324
31. Law P K, Bertorini T E, Goodwin T G et al. Dystrophin production induced by myoblast transfer therapy in Duchenne muscular dystrophy. Lancet. 1990; 336:114-115
32. Gussoni E, Pavlath G K, Lanctot A M et al. Normal dystrophin transcripts detected in Duchenne muscular dystrophy patients after myoblast transplantation. Nature. 1992; 356:435-438
33. Huard J, Bouchard J P, Roy R et al. Human myoblast transplantation: preliminary results of 4 cases. Muscle Nerve. 1992; 15:550-560
34. Mendell J R, Kissel J T, Amato A A et al. Myoblast transfer in the treatment of Duchenne's muscular dystrophy. N Engl J Med. 1995; 333:832-838
35. Morandi L, Bernasconi P, Gebbia M et al. Lack of mRNA and dystrophin expression in DMD patients three months after myoblast transfer. Neuromuscul Disord. 1995; 5:291-295
36. Karpati G, Ajdukovic D, Arnold D et al. Myoblast transfer in Duchenne muscular dystrophy. Ann Neurol. 1993; 34:8-17
37. Bajek A, Porowinska D, Kloskowski T et al. Cell therapy in Duchenne muscular dystrophy treatment: clinical trials overview. Crit Rev Eukaryot Gene Expr. 2015; 25:1-11
38. Montarras D, Morgan J, Collins C et al. Direct isolation of satellite cells for skeletal muscle regeneration. Science. 2005; 309:2064-2067
39. Gilbert P M, Havenstrite K L, Magnusson K E et al. Substrate elasticity regulates skeletal muscle stem cell self-renewal in culture. Science. 2010; 329:1078-1081
40. Xu X, Wilschut K J, Kouklis G et al. Human Satellite Cell Transplantation and Regeneration from Diverse Skeletal Muscles. Stem Cell Reports. 2015; 5:419-434
41. Charville G W, Cheung T H, Yoo B et al. Ex Vivo Expansion and In Vivo Self-Renewal of Human Muscle Stem Cells. Stem Cell Reports. 2015; 5:621-632
42. Meng J, Adkin C F, Xu S W et al. Contribution of human muscle-derived cells to skeletal muscle regeneration in dystrophic host mice. PLoS One. 2011; 6:e17454
43. Chirieleison S M, Feduska J M, Schugar R C et al. Human muscle-derived cell populations isolated by differential adhesion rates: phenotype and contribution to skeletal muscle regeneration in Mdx/SCID mice. Tissue Eng Part A. 2012; 18:232-241
44. Price F D, Kuroda K, Rudnicki M A. Stem cell based therapies to treat muscular dystrophy. Biochim Biophys Acta. 2007; 1772:272-283
45. Tedesco F S, Dellavalle A, Diaz-Manera J et al. Repairing skeletal muscle: regenerative potential of skeletal muscle stem cells. J Clin Invest. 2010; 120:11-19
46. Wilschut K J, Ling V B, Bernstein H S. Concise review: stem cell therapy for muscular dystrophies. Stem Cells Transl Med. 2012; 1:833-842
47. Nesmith A P, Wagner M A, Pasqualini F S et al. A human in vitro model of Duchenne muscular dystrophy muscle formation and contractility. J Cell Biol. 2016; 215:47-56

### EXAMPLES

### Construction of Homologous Recombination Donor Vector Containing PolyA Signal Cassette

For the Donor vector shown in FIG. 3 AAV2-MSC2 vector (6954, Addgene) was used as a backbone vector. The insertion cassette, which contained the PolyA signals and selectable GFP marker followed by a 2A self-cleaving peptide and puromycin resistance gene, was assembled using standard cloning techniques. Site-specific insertion was mediated by incorporating homologous arms flanking the insertion cassette. 5′-homologous arms (500 bp) were PCR amplified using high fidelity DNA polymerase (Platinum Pfx DNA Polymerase, Invitrogen, Carlsbad, Calif., USA), and a 3′-homologous arm (184 bp) was synthesized by GenScript (Piscataway, N.J., USA). Two single gRNA transcription units were also synthesized and cloned upstream of the 5-homologous arm. The whole cassette was cloned between NheI and MluI sites of the AAV2-MSC2 vector. This donor was used in this study for the generation of iPSC clones. The puromycin coding sequencing can be removed by BsiWI without affecting the GFP expression to generate a donor so that the remaining cassette (3.71 kb) flanked by inverted terminal repeats (ITRs) can be packaged to AAV for in vivo application and the expression of GFP can be tracked for transduction efficiency (FIG. 4). As shown in FIG. 5, for clinical applications, the GFP-expressing cassette can also be removed by Hind III to avoid unnecessary ectopic DNAs.

DM1 iPSC Transfection and Clone Selection

The normal control iPSC and DM1 iPSC (DM-03) lines were established using protocols described in Xia et al., “Generation of neural cells from DM1 induced pluripotent stem cells as cellular model for the study of central nervous system neuropathogenesis.” Cell. Reprogram. 15, 166-177 (2013), which is hereby incorporated by reference for all purposes. For transfection, DM1 iPSCs (DM-03) were passed as small colonies using Gentle Cell Dissociation Reagent (STEMCELL Tehnologies) the day before transfection on a Vitronectin-coated 6-well plate in mTeSR-E8medium (STEMCELL Technologies). Transfection was conducted with Lipfectamine LTX reagent with PLUS Reagent. Briefly, 1 ug of each donor vector and SpCas9 nickase plasmid was mixed with 3 uL plus reagent and then with 12 ul Lipfectamine. They were incubated at room temperature for 5 min and then the complex was added dropwise to 1 well of cultured cells in a 6-well plate (2 mL in each well). Medium was changed 24 hours later. Puromycin selection was started 48 hours after transfection at 0.4 ug/ml. Selection was continued until individual clones were large enough for isolation. The GFP-positive and puromycin-resistant clones were selected and subjected to FISH. Intranuclear RNA CUG foci-negative clones were identified for further characterization.

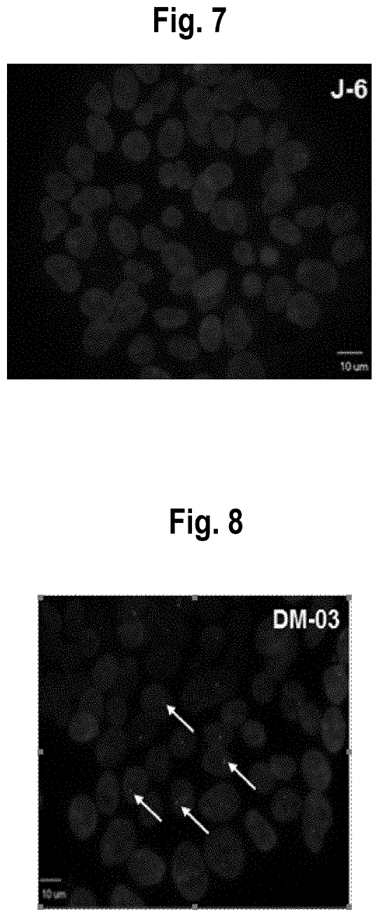

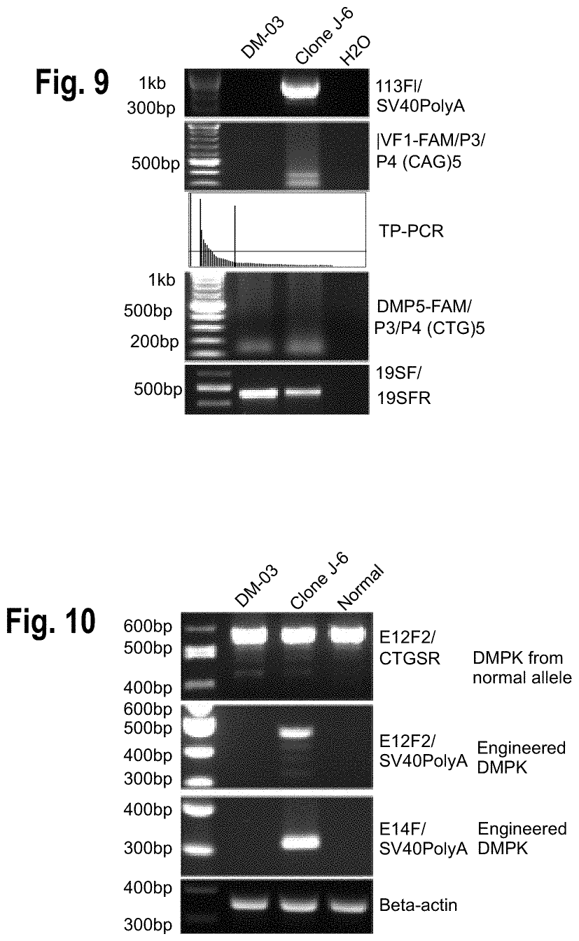

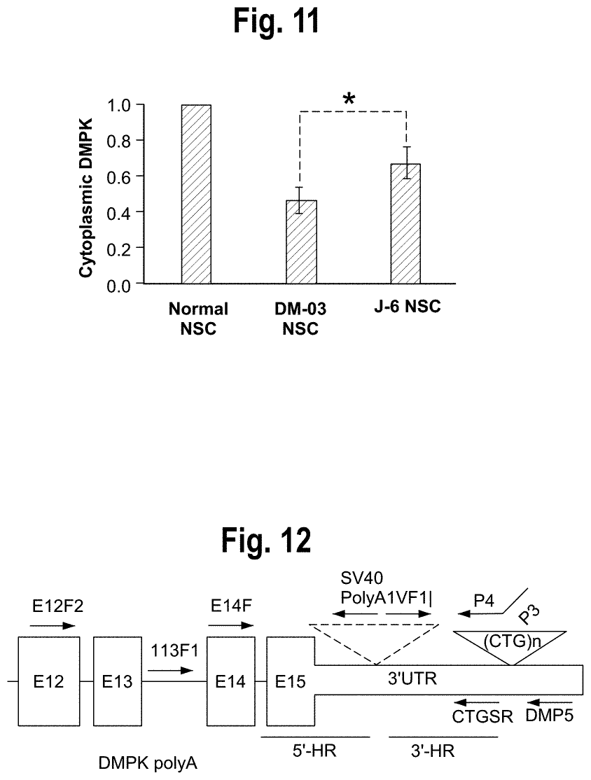

FIGS. 7 and 8 compare the images of genome edited iPSCs carrying the DM1 mutation (FIG. 7) and unedited iPSCs carrying the DM1 mutation (FIG. 8). The genome edited iPSCs (J-6) in FIG. 7 showed a complete loss of intranuclear RNA CUG repeat foci in each cell within a colony that might be derived from a single clone (no bright spots). In contrast, the unmodified parental DM1 iPSCs (DM-03) in FIG. 8 showed intranuclear RNA CUG repeat foci (bright spots identified by arrows). Immunofluorescent staining (not shown) showed that the genome edited cells maintained expression of pluripotent stem cell markers. Turning to FIG. 9, genotyping by junctional PCR and TP-PCR showed the correct insertion of the PolyA cassettes in the 3′ UTR. TP-PCR from IVF1-FAM/P3P4 (CAG)₅ further confirmed the presence of expanded CTG repeats and the identity of clone J-6 from parental DM-03 iPSC. Turning to FIG. 10, RT-PCR showed the expression of normal DMPK transcripts in clone J-6, suggesting that the normal allele was not affected. PCR products from E12F2/SV40PolyA showed the edited DMPK mRNA with SV40PolyA in the J-6 clone had the same splicing pattern as wild-type DMPK mRNA. Both the top and bottom bands of the wild-type DMPK and DMPK with SV40PolyA were verified by Sanger sequencing, and both showed the inclusion or exclusion of exons 13 and 14 (data not shown.) FIG. 11 is the results of a quantitative RT-PCR of cytoplasmic DMPK RNA showing significantly increased cytoplasmic DMPK RNA in NSCs derived from genome-edited J-6 iPSCs compared to parental DM-03 derived NSCs. *p<0.01 by Student's t test. FIG. 12 is a schematic view of the primer positions. Additional details may be found, for example, in Wang et al., “Therapeutic Genome Editing for Myotonic Dystrophy Type 1 Using CRISPR/Cas9” Mol. Ther. Vol. 26 No. 11., which is hereby incorporated by reference for all purposes.

### Skeletal Muscle Differentiation

Skeletal muscle differentiation was first performed by a quick induction method (7 days) according to the manufacturer's protocol (QMS-SeV, Elixirgen Scientific, Baltimore, Md., USA) which is based on a published technology of ectopic expression of a demethylase (JMJD3) and a linear-defining transcription factor (MYOD1). See, e.g., Seznec, H., et al. (2001). Mice transgenic for the human myo-tonic dystrophy region with expanded CTG repeats display muscular and brain ab-normalities. Hum. Mol. Genet. 10, 2717-2726, which is hereby incorporated by reference for all purposes. The protocol generated a high percentage of myosin-positive myocytes in 7 days. To isolate skeletal muscle progenitor cells, a modified version of the protocol described in Chal et al. (2016). Generation of human muscle fibers and satellite-like cells from human pluripotent stem cells in vitro. Nat. Protoc. 11, 1833-1850, was used. Briefly, iPSCs were harvested by TrypLE treatment at 37° C. for 7 min and resuspended as single cells in mTdSR E8 medium supplemented with 10 uM Rock inhibitor (Y-27362). 2.8×10⁵ iPSCs were plated into 6-well plates and 8-well chamber slides coated with Matrigel (Corning Life Sciences). Differentiation was initiated when the wells reached 20% confluency using sequential induction with WNT activator (4423, CHIR99021, 3 uM, Tocris Bioscience), BMP inhibitor (04-0074m LDN193189, 0.5 uM, Stemgent), fibroblast growth factor 2 (FGF2, 450-33, 20 ng/mL, Pepro Tech), hepatocyte growth factor (HGF, 315-23, 10 ng/ml, PeproTech). On day 20, skeletal muscle progenitor cells in 6-well plates were isolated by TrypLE and 0.05% trypsin and EDTA treatment of the wells and cultured in Matrigel-coated 8-well plates in Skeletal Muscle Cell Growth Media (SKGM-2, CC-3245, Lonza.) The expression of skeletal muscle progenitor cell markers (PAX3 and PAX7) was monitored by IF staining. On day 30, differentiation in 8-well chamber slides was subjected to FISH and IF for myosin heavy chain (MHC).

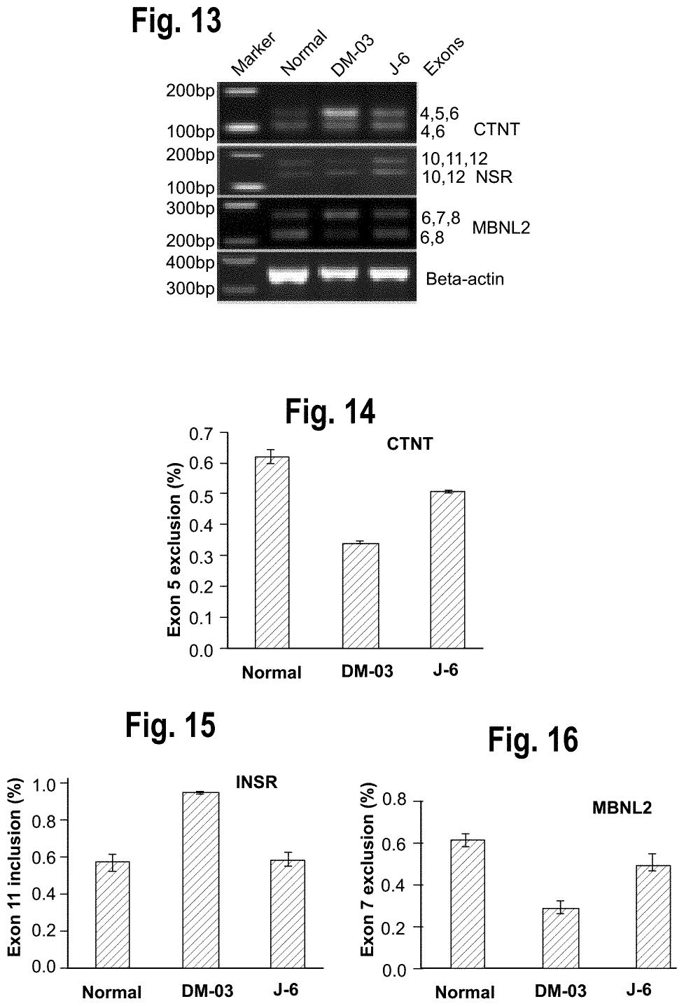

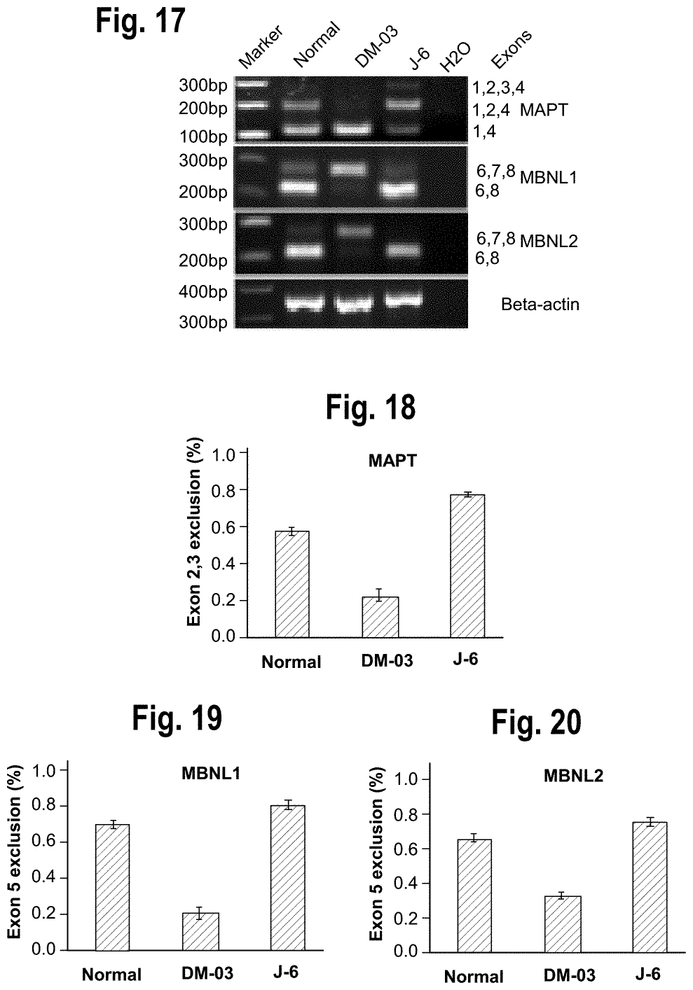

After differentiation in cardiomyocytes, intranuclear RNA CUG repeat foci were detected in cardiomyocytes derived from parental DM-03 iPSCs but not in cardiomyocytes derived from the genome-edited J-6 iPSCs. (Image not shown.) An agarose gel image (FIG. 13) and quantitative analysis (FIGS. 14-16) showed the reversal of aberrant splicing patterns in cardiac troponin T (CTNT), insulin receptor (INSR), and muscleblind-like 2 (MBNL2) in cardiomyocytes. The iPSCs were also differentiated into neural stem cells (NSCs) which similarly showed foci (bright spots identified with arrows) in NSCs derived from DM-03 iPSCs but no foci in the NSCs derived from genome-edited J-6 iPSCs (Image not shown). An agarose gel image (FIG. 17) and quantitative analysis (FIGS. 18-20) showed the reversal of aberrant splicing patterns in microtubule-associated protein tau (MAPT) and MBNL1, 2 in NSCs-derived cardiomyocytes. (Data are represented as mean+/−SEM.) NSCs were further differentiated into forebrain neurons and again they showed the loss of intranuclear RNA CUG repeat foci and reversal of known aberrant alternative splicing of MAPT, MBNL1, 2, Sarcoplasmic/endoplasmic reticulum Ca2⁺ ATPase 1 (SERCA 1) and INSR. (Data not shown.) Additional details may be found, for example, in Wang et al., “Therapeutic Genome Editing for Myotonic Dystrophy Type 1 Using CRISPR/Cas9” Mol. Ther. Vol. 26 No. 11., which is previously incorporate by reference.

What is claimed is:

1. A method for editing a CTG repeat mutation in the Dystrophia Myotonica protein kinase (DMPK) gene that results in myotonic dystrophy type 1 (DM1) comprising inserting a polyadenylation signal (PAS) in an insertion site in the 3′ Untranslated Region (3′-UTR) of the DMPK gene, wherein the insertion site is upstream of the CTG repeats, to produce an edited DMPK gene.

2. The method of claim 1, wherein the PAS is inserted by way of an insertion cassette comprising at least one PAS flanked by a first DNA sequence that is homologous to a portion of the DMPK gene that is 3′ of the insertion site and a second DNA sequence that is homologous to a portion of the DMPK gene that is 5′ of the insertion site.

3. The method of claim 2, wherein a first insertion cassette comprises the PAS and a pair of gRNAs as flanking sequences, and a second insertion cassette is an SpCas9 cassette.

4. The method of claim 2, wherein the DMPK gene is in a viable cell.

5. The method of claim 4, wherein the insertion cassette is part of a donor vector.

6. The method of claim 5, wherein the donor vector is an AAV-based donor vector.

7. The method of claim 4 wherein the viable cell is inside of a living subject.

8. The method of claim 7 wherein the living subject is a human being who has been diagnosed with DM1 or who has been identified as carrying the DM1 mutation.

9. The method of claim 4 wherein the cell is an iPSC cell.

10. The method of claim 9 wherein the iPSC cell has been cultured from cells obtained from a living subject.

11. The method of claim 10 further comprising differentiating the iPSC cell into genome edited skeletal myogenic progenitor cells (SMPCs) after the PAS is inserted into the 3′ UTR of the DMPK gene.

12. The method of claim 11 further comprising transplanting the genome edited SMPCs into the subject.

13. The method of claim 12 further comprising delivering to the subject, IGF-1 producing monocyte cells.

14. A Dystrophia Myotonica protein kinase (DMPK) gene having a CTG repeat mutation and a polyadenylation signal inserted in an insertion site in the 3′ Untranslated Region (UTR) of the DMPK gene upstream of the CTG repeats.

15. A viable cell comprising a Dystrophia Myotonica protein kinase (DMPK) gene having a CTG repeat mutation and a polyadenylation signal inserted in an insertion site in the 3′ Untranslated Region (3′-UTR) of the DMPK gene upstream of the CTG repeats.

16. The cell of claim 15 wherein the cell is an iPSC cell.

17. The cell of claim 15 wherein the iPSC cell has been derived from a cell sample taken from living subject.

18. The cell of claim 15 wherein the cell is a genome edited skeletal myogenic progenitor cell (SMPC).

19. The cell of claim 18 wherein the genome edited SMPC was differentiated from a genome edited iPSC cell.

20. The cell of claim 15 wherein the SMPC was genome edited in vivo.

21. A method of treating myotonic dystrophy type 1 (DM1) in a subject wherein the DM1 is caused by a CTG repeat mutation in the Dystrophia Myotonica protein kinase (DMPK) gene comprising editing the DM1 mutation by inserting a polyadenylation signal (PAS) into an insertion site in the 3′ Untranslated Region (UTR) of the mutant DMPK gene upstream of the CTG repeats.

22. The method of claim 21 further comprising injecting into the subject, a vector comprising an insertion cassette comprising at least one PAS flanked by a first DNA sequence that is homologous to a portion of the DMPK gene that is 3′ of the insertion site and a second DNA sequence that is homologous to a portion of the DMPK gene that is 5′ of the insertion site.

23. The method of claim 21 further comprising:
obtaining a cell sample from the subject;
obtaining an iPSC cell from the cell sample,
editing the genome of the iPSC cell by delivering an insertion cassette comprising at least one PAS flanked by a first DNA sequence that is homologous to a portion of the DMPK gene that is 3′ of the insertion site and a second DNA sequence that is homologous to a portion of the DMPK gene that is 5′ of the insertion site under sufficient conditions that the PAS is inserted into the insertion site thereby producing genome-edited iPSC cells;
differentiating the genome-edited iPSC cells into skeletal myogenic progenitor cells (SMPCs); and
transplanting the SMPCs into the subject.

24. The method of claim 23 further comprising delivering IGF-1 producing monocyte cells to the subject.
